SEQUENCE LISTING (2 sequences)

SEQ ID NO: 1 - RNA, 10 nt, Artificial Sequence
  a RNA stem-loop mimic of the sacrin-ricin loop
cgcgagagcg

SEQ ID NO: 2 - DNA, 14 bp, Artificial Sequence
  a RNA/DNA hybrid stem-loop A-14 2dA
cgcgcgagagcgcg

(19) United States
(12) Patent Application Publication
(10) Pub. No.: US 2011/0136106 A1
(43) Pub. Date: Jun. 9, 2011

(54) METHODS FOR DETECTING RICIN AND RELATED COMPOUNDS AND USES THEREOF

(76) Inventor: Vern L. Schramm, New Rochelle, NY (US)

(21) Appl. No.: 12/737,464

(22) PCT Filed: Jul. 14, 2009
(86) PCT No.: PCT/US09/04075

§ 371 Date: Feb. 24, 2011

Related U.S. Application Data
(60) Provisional application No. 61/135,558, filed on Jul. 22, 2008.

(51) Int. Cl. C12Q 1/68

(52) U.S. Cl. 435/6.1

(57) ABSTRACT

The present invention is directed to methods for detecting the presence of ricin and compounds that catalyze the release of adenine from nucleic acids or other biological materials and for screening for inhibitors of such compounds.

### CROSS-REFERENCE TO RELATED APPLICATION

This application claims the benefit of U.S. Provisional Patent Application No. 61/135,558, filed on Jul. 22, 2008, the content of which is hereby incorporated by reference.

### STATEMENT OF GOVERNMENT SUPPORT

The invention disclosed herein was made with U.S. Government support under grant number CA72444 from the National Institutes of Health. Accordingly, the U.S. Government has certain rights in this invention.

### FIELD OF THE INVENTION

The present invention generally relates to methods for detecting and quantifying ricin and related compounds and for identifying inhibitors of these compounds.

### BACKGROUND OF THE INVENTION

Throughout this application various publications are referred to by Arabic numerals in brackets. Full citations for these references may be found at the end of the specification immediately preceding the claims. The disclosures of these publications are hereby incorporated by reference in their entireties into the subject application to more fully describe the art to which the subject application pertains.

Ricin, a potent toxin and potential bioterrorism threat, is a ribosome-inactivating protein (RIP) abundant in castor beans. It is toxic by inhalation, oral, and intravenous exposure [1]. The ricin type II RIP is comprised of a toxic A-chain (RTA) and lectin B-chain (RTB) linked by a single disulfide bond. The entry of ricin into cells is mediated via RTB binding cell surface galactose receptors [2]. The endocytosed toxin undergoes retrograde transport from the golgi to the endoplasmic reticulum where disulfide bond cleavage occurs and RTA is chaperoned into the cytosol [3-5]. Within the cytosol, RTA binds the 28S portion of the 60S ribosomal subunit and catalyzes the hydrolytic depurination of _{A4324,} the first adenosine of the conserved GAGA loop portion of the sarcin-ricin loop (SRL) [6]. Depurination of the SRL causes the loss of elongation factor binding, inhibition of protein synthesis, and cellular death.

The sensitive quantification of adenine release from ricin substrates is essential in order to detect ricin and related toxins. Such a method is also of utility in establishing the kinetic mechanism of depurinating toxins in ribosome and nucleic acid catalysis. Current methods of quantifying adenine from ribosome-inactivating protein (RIP) depurination reactions include separation of adenine by HPLC (with or without fluorescent derivatization) and a continuous colorimetric enzyme coupled assay [7-9]. However, present methods lack the sensitivity to measure the initial rates of ribosome depurination by RIP catalysis and thus a gel based assay is the standard method used to determine kinetic parameters on radiolabeled ribosomes where the depurinated product is chemically cleaved at the site of depurination and the 28S product fragments are analyzed by PAGE [10, 11].

Accordingly, there is a compelling need for a method that measures minute amounts of ricin and can quantitate it before toxic levels are reached. The present invention satisfies this need.

### SUMMARY OF THE INVENTION

The present invention is directed to methods for detecting the presence of ricin and compounds that catalyze depurination of adenosine to release adenine from nucleic acids. The present invention is further directed to a method for screening for inhibitors of ricin and of compounds that catalyze depurination of adenosine to release adenine from nucleic acids.

### BRIEF DESCRIPTION OF THE FIGURES

FIG. 1. Diagram of one embodiment of the claimed method. Adenine released from the ricin A-chain (RTA) depurination of stem-loop or ribosome (1) is converted to AMP with adenine phosphoribosyl transferase (APRTase) and then to ATP with pyruvate orthophosphate dikinase (PPDK) where the ATP drives the luciferase enzymatic reaction to produce luminescence (2). At neutral pH, steps 1 and 2 are combined in one reaction for direct adenine assays.

FIGS. 2A-2B. Adenine standards: A) Linear fit for luminescence versus increasing quantities of adenine from 1-2,000 picomoles. Adenine was assayed discontinuously by preparing the standards in 25 μL of water with 25 μL of 2x activated coupling buffer. A 20 μL aliquot was assayed for ATP concentration with a D-luciferin/luciferase reagent (ATPlite). B) Continuous luminescent assay of increasing concentrations of adenine. From bottom to top are blank, 0.5, 1, 2, 4, and 8 picomoles of adenine assayed individually in a total volume of 50 μL of 1x continuous assay buffer in a 96-well plate. Upon the addition of adenine, the plate was shaken for 10 seconds followed by 2 minutes of kinetic acquisition with 1 data point per second.

FIGS. 3A-3D. Discontinuous adenine-luciferase assay format kinetic curve fits for RTA catalysis of stem-loop RNA A-10 (A) and A-14-2dA (B) at pH 4.0. Kinetic curve fits for RTA catalysis of 60S yeast (C) and 80S rabbit (D) ribosome at pH 7.4 in 20 mM tris-HC1, 25 mM KC1, and 5 mM MgC1₂.

FIGS. 4A-4D. Continuous assay measurements. A) initial rate slopes (lumens/second) of increasing concentrations of 80S rabbit ribosome with 33 pM RTA and B) kinetic curve fit of the calculated kinetic rate from (A) versus 80S ribosome concentration. C) Kinetic curve fit for RTA catalysis of 60S yeast ribosome measured in the continuous adenine-luciferase assay. D) The detection of 7 femptomoles of RTA with increasing picomoles of 80S rabbit ribosome in 50 μL reactions (bottom to top: control, 1 , 2 , 4, and 8 picomoles).

### DETAILED DESCRIPTION OF THE INVENTION

The present invention provides a method for detecting the presence of a compound that catalyzes release of adenine from a nucleic acid, the method comprising (a) converting adenine released by catalysis of a nucleic acid by the compound to adenosine monophosphate (AMP) using adenosine phosphoribosyl transferase, and (b) converting the AMP formed in step (a) to adenosine triphosphate (ATP) using pyruvate orthophosphate dikinase, wherein the presence of ATP is indicative of the presence of the compound.

In one embodiment, the amount of ATP formed in step (b) is proportional to the amount of the compound. Numerous methods are known in the art for measuring ATP. Examples of such methods are described in U.S. Pat. Nos. 7,122,333, 7,083,911, and 5,916802, the contents of which are hereby incorporated by reference into the subject application.

In a preferred embodiment, ATP is detected using a luciferase. Luciferase is a common bioluminescent enzyme. Organisms from which luciferase can be obtained include, but are not limited to fireflies, bacteria (e.g. Vibrio fischeri), jellyfish (e.g. Aequorea victoria), beetles, and squid. In the preferred embodiment of the present invention, the luciferase is firefly luciferase.

The nucleic acid can be DNA or RNA. In one embodiment, the RNA is associated with a ribosome.

In one embodiment of the subject method, the compound is a ribosome-inactivating protein (RIP). Numerous ribosome-inactivating proteins are known in the art, and include, but are not limited to, ricin, cinnamomin, saporin, abrin, and modeccin. In the preferred embodiment of the invention, the ribosome-inactivating protein is ricin.

The present invention further provides a method for detecting the presence of ricin comprising (a) converting adenine released by catalysis of a nucleic acid by ricin to AMP using adenosine phosphoribosyl transferase, and (b) converting the AMP formed in step (a) to ATP using pyruvate orthophosphate dikinase, wherein the presence of ATP is indicative of the presence of ricin.

In one embodiment, the amount of ATP formed in step (b) is proportional to the amount of ricin. Preferably, ATP is detected using a luciferase. In the preferred embodiment of the present invention, the luciferase is firefly luciferase.

Preferably, the nucleic acid is RNA. The RNA can be associated with a ribosome.

The present invention further provides a method for screening for an inhibitor of a compound that catalyzes release of adenine from a nucleic acid, the method comprising (a) contacting nucleic acid with the compound in the presence and in the absence of a potential inhibitor of the compound, (b) converting adenine released by catalysis of the nucleic acid by the compound to AMP using adenosine phosphoribosyl transferase, (c) converting the AMP formed in step (b) to ATP using pyruvate orthophosphate dikinase, and (d) measuring the amount of ATP formed in step (c), wherein a reduction in the amount of ATP formed in the presence of the potential inhibitor compared to the amount of ATP formed in the absence of the potential inhibitor indicates that the potential inhibitor is an inhibitor of the compound, or wherein a lack of reduction in the amount of ATP formed in the presence of the potential inhibitor compared to the amount of ATP formed in the absence of the potential inhibitor indicates that the potential inhibitor is not an inhibitor of the compound.

In one embodiment, the amount of ATP formed in step (c) is proportional to the amount of the compound. Preferably, ATP is detected using a luciferase. Preferably, the luciferase is firefly luciferase.

The nucleic acid can be DNA or RNA. The RNA can be associated with a ribosome.

In an embodiment of the subject method, the compound is a ribosome-inactivating protein (RIP). In the preferred embodiment of the invention, the ribosome-inactivating protein is ricin.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

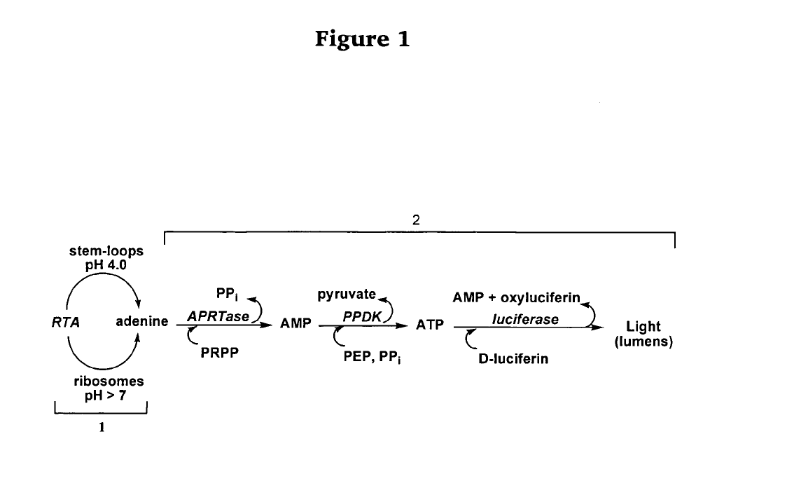

An enzyme coupled assay that can quantify picomole amounts of adenine in order to directly measure RTA catalysis of ribosomes is described. The conversion of adenine to AMP by adenosine phosphoribosyl transferase (APRTase) has been reported as the first step in an adenine colorimetric assay for the detection of RIP activity with nanomole sensitivity for adenine [9]. For measuring AMP and ATP levels with sub-femtomole sensitivity, a pyruvate orthophosphate dikinase (PPDK) cycling reaction with firefly luciferase has been previously described [12]. The subject assay combines APRTase and PPDK in a one pot reaction where free adenine is converted to AMP with APRTase and then to ATP with PPDK where the ATP can be assayed by a firefly luciferase ATP assay kit (FIG. 1). Continuous measurements are accomplished in a 96-well plate format by combining the luciferase reagent with APRTase/PPDK (adenine to ATP) coupling enzymes.

The depurination of 80S rabbit reticulocyte ribosome and 60S yeast ribosome by RTA was investigated using the adenine-luciferase coupled assay to calculate initial rate kinetic parameters in continuous and discontinuous adenine formation formats. A-10, a RNA stem-loop (5′- CGCGAGAGCG-3′) (SEQ ID NO:1) mimic of the SRL was assayed at pH 4.0 with RTA to compare kinetic parameters previously obtained with an HPLC based assay [8]. A RNA/DNA hybrid stem-loop A-14 2dA (5′-CGCGCGdAGAGCGCG-3′) (SEQ ID NO:2), where _{d}A is deoxyadenylate, was also assayed and the kinetic parameters are consistent with previous reports of deoxyadenosine at the depurination site in RNA stem-loop oligonucleotides enhancing RTA catalytic turnover [13].

Materials and Methods

Ricin Toxin A-chain (RTA) was purchased from Sigma. The plasmid containing the PPDK clone from Clostridium symbiosum was obtained from Dr. Debra Dunaway-Mariano, University of New Mexico [14,15]. 60S yeast ribosome was obtained from Dr. Maitra (Albert Einstein College, Bronx N.Y.). Rabbit reticulocyte lysate (untreated) was purchased from Promega (Madison, Wis.). Oligonucleotides A-10 and A-14 2dA were purchased from Dharmacon (Lafayette, Colo.). Firefly luciferase ATP assay kit (ATPlite) was purchased from Perkin Elmer (Waltham, Mass.). Phosphatase inhibitors were purchased from Roche Applied Science (Indianapolis, Ind.). RNase inhibitor (SuperRNasin) was purchased from Ambion (Austin, Tex.). Buffers and enzyme preparations were checked for RNase activity using RNaseAlert from Ambion (Austin, Tex.). DEPC treated water was used for all enzymatic reactions. All other reagents used were purchased in the highest purity available from Fisher Scientific (Pittsburgh, Pa.) or Aldrich Chemical Corp. (Ashland, Mass.). Spectrophotometric assays and concentrations of adenine, ribosomes, and oligonucleotides were measured using a Varian Cary 100 diode array spectrophotometer. Luminescence was measured on a GloMax 96-well luminometer from Promega (Madison, Wis.).

Adenine to ATP conversion buffer. 50 mL of 2x coupling buffer [100 mM Tris-acetate pH 7.7, 2 mM phosphoenolpyruvic acid, 2 mM sodium pyrophosphate, 2 mM 5-phospho-D-ribosyl-1-pyrophosphate (PRPP), 15 mM NH₄SO₄, and 15 mM (NH₄)₂MoO₄] was prepared in DEPC treated water with phosphatase inhibitors. 7 mL of charcoal/cellulose (1:5) powder was suspended in DEPC water and packed into a PD10 column with 100 mL of water at 2 mL/min. The 2x coupling buffer was passed through the column at 2 ml/min in order to remove adenine, AMP, and ATP contamination. The eluant was filtered (0.2 μm) and stored in 1 mL aliquots at −80° C.

The enzymatic activity (units) of PPDK was determined spectrophotometrically at 340 nM by coupling pyruvate formation to a lactate dehydrogenase (18 units/ml)-NADH (0.2 mM) assay with 50 μM AMP added to start the reaction at 20° C. in 1x coupling buffer in water with 5 mM MgSO₄. APRTase activity (units) was determined in identical conditions without AMP, with excess units of PPDK present, and 50 μM of adenine to start the reaction.

Prior to use in catalytic assays, the 2x coupling buffer (1 mL) was thawed and 10 mM MgSO₄, 2 units of APRTase, 4 units of PPDK , and RNAse inhibitor were added in order to constitute the 2x activated coupling buffer.

Expression and purification of Clostridium symbiosum PPDK. PPDK was expressed and purified as described previously with some modification [14, 15]. The plasmid encoding PPDK from Clostridium symbiosum was expressed in Escherichia coli JM101 cells grown in LB medium with 12.5 μM tetracycline to an A₆₀₀ of 1.4. The cells were harvested by centrifugation at 2,000 g for 30 minutes at 4° C. The cell pellet was suspended in buffer (20 mM imidazole, 2.5 mM Na₂EDTA, 2 mM DTT, 75 mM KC1, pH 6.8 with protease inhibitors) and the cells were disrupted by French press followed by centrifugation at 17,000 g for 1 hour at 4° C. After streptomycin sulfate, ammonium sulfate (50-70%) and DEAE cellulose column purification steps, PPDK was dialyzed (20 mM imidazole, 2.5 mM Na₂EDTA, 1 mM DTT, 88 mM KC1, pH 6.4) at 4° C. and loaded onto a Sephacryl S-200 column (GE Science) and eluted in dialysis buffer [14]. Pure PPDK fractions were identified by SDS-PAGE. Glycerol to 10% (final volume ratio) was added and aliquots were flash frozen in dry ice/ethanol and stored at −80° C.

Expression and Purification of S. cerevisiae APRTase (scAPRTase). scAPRTase was expressed and purified as described previously with some modification [16]. In brief, E. coli (B25) cells carrying N-terminal 6 x His scAPRTase expression vector were grown at 37° C. in LB containing ampicillin (100 μg/mL), kanamycin (25 μg/mL) and sodium phosphate (10 mM, pH=7.5). Expression of scAPRTase was induced by 2 mM isopropyl α-D-thiogalactopyranoside when A₆₀₀ of cultures reached 0.7. Cells were harvested after 5 h at 37° C. (centrifugation at 5000 g for 20 min at)4° . Cell pellets were suspended in buffer A (50 mM Tris, 20 mM KCl, 5 mM MgCl₂, pH=7.4) and then disrupted using a French press, followed by centrifugation at 20,000 g for 20 min. The supernatant was fractionated by ammoniums sulfate precipitation (35% -70% fraction). The resulting pellets were then dialyzed against buffer A containing 50 mM imidazole and loaded onto a 25 mL Ni Sepharose High Performance (GE Healthcare, Piscataway) His-tag affinity column. The column was washed with buffer A containing 50 mM imidazole and scAPRTase was eluted with 50-300 mM gradient imidazole. After adding 10% glycerol (final volume ratio), APRTase fractions (PAGE analysis) were combined and concentrated. This crude APRTase was subject to Mono-Q ion-exchange chromatography with a linear elution gradient of 20-300 mM KCl. The APRTase fractions (PAGE analysis) were added with 10% glycerol (final volume ratio), concentrated and then purified by Superdex75 (GE Science) with Buffer A containing 10% glycerol (GE Science). The APRTase fractions were concentrated to give −80 ° C. stock solution (10 mg / ml). The four-step purification is needed to remove trace DNAase/RNAase activites in scAPRTase.

Ribosome isolation. 80S eukaryotic ribosomes were purified from untreated rabbit reticulocyte lysate. 500 μl of lysate was layered onto 250 μl of sucrose cushion (1 M sucrose, 20 mM Tris-HCl pH 7.5, 500 mM KC1, 2.5 mM MgCl₂, 0.1 mM EDTA, and 0.5 mM DTT) and centrifuged at 100,000 rpm in a TLA 120.2 rotor for 2 hours at 4° C. The glassy pellet was washed twice on ice with ribosome storage buffer (20 mM Hepes-KOH pH 7.7, 100 mM KCl, 5 mM MgCl₂, 0.1 mM EDTA, 250 mM sucrose, and 1 mM DTT) and re-suspended in a minimal volume of the same buffer. The 80S ribosome was flash frozen in dry ice/ethanol and stored at −80° C.

Prior to use in catalytic assays, 60S and 80S ribosome were buffer exchanged into ribosome reaction buffer (20 mM Tris-HC1 pH 7.4, 25 mM KC1, and 5 mM MgCl₂) with a G-25 microcentrifuge desalting spin column at 4° C. (Pierce). Concentrations of 60S and 80S ribosome preparations were calculated by absorbance at 260 nm [17]. Active concentrations of 60S and 80S ribosomes were determined by complete depurination by RTA in kinetic assays (materials and methods).

Discontinous assay kinetics. Varying concentrations of substrate A-10 or A-14-2dA were incubated at 37° C. for 5 minutes in acidic RTA buffer (10 mM potassium citrate-KOH and 1 mM EDTA at pH 4.0). Reactions were initiated by the addition of RTA at concentrations typically between 1-20 nM to a total reaction volume of 25 μL. The reactions were quenched at timed intervals with 25 μL of 2x activated coupling buffer to give <15% product formation. In parallel, five adenine standards and three controls (buffer, buffer + substrate, and RTA + buffer) were made in acidic RTA buffer diluted with 2x activated coupling buffer in volumes identical to the substrate assay condition. The quenched RTA reactions, standards, and control solutions were incubated at room temperature for 2 minutes. 20 μL aliquots were assayed for ATP concentration with a D-luciferin/luciferase ATP assay mixture (ATPlite) according to the manufactures protocol. Luminescence (RLU) was converted to pMoles of adenine from the standard curve fit (corrected for background from controls) and initial rate kinetics were fit to the Michaelis-Menten equation for the calculation of substrate k_{cat} and Kₘ.

For discontinuous ribosome assays, 60S or 80S ribosomes were incubated for 5 minutes in pH 7.4 ribosome reaction buffer. Reactions were initiated by the addition of RTA at concentrations between 30-70 pM in a total reaction volume of 25 μL. The reactions were quenched with 60 mM HC1 at timed intervals to give <15% product formation, incubated on ice for 2 minutes, and brought to pH near 7.0 with 60 mM KOH. An equal volume of 2x activated coupling buffer was added to the quenched reactions and incubated at room temperature for 2 minutes. Adenine standards and controls (as described for stem-loop assays) were prepared in ribosome reaction buffer and were quenched in identical buffer conditions as the ribosome RTA catalytic reactions. ATP concentration of the aliquots was measured with D-luciferin/luciferase mixture (ATPlite) as described above. Ribosome concentration was determined by depurinating (to completion) two stock concentrations with 500 nM RTA and comparing the final luminescence to the adenine standard curve fit. PicoMoles of adenine released during the assay and initial rate kinetics were calculated identically to stem-loop assays after appropriate background corrections were made from controls.

Continuous assay kinetics. 200 μl of ATPlite was added to 1 mL of 2x activated coupling buffer comprising the 2x continuous coupling buffer. The buffer was equilibrated to room temperature (20° C.) for 10 minutes prior to assays. In 96-well plate format, varying concentrations of ribosome were individually assayed in kinetic acquisition mode of the luminometer in a total reaction volume of 50 μL in 1x continuous assay buffer. RTA was prepared in ice cold ribosome reaction buffer with a single dilution from stock and equilibrated to room temperature before assays. A one minute blank reading for each ribosome concentration was made before the addition of RTA (30-70 pM) to ensure a stable luminescent signal. Luminescence was monitored for several minutes after RTA addition. Adenine standards in the concentration range of the assay reactions were preformed before and after continuous ribosome assays. Ribosome concentration was determined by depurinating (to completion) two stock concentrations with 500 nM RTA and comparing the final luminescence to the adenine standard curve fit. The initial rate of adenine formation from the catalytic reactions were calculated by converting luminescent rate (lumens/second) to enzymatic rate (pMoles of adenine/minute/pMoles enzyme) using the adenine standard curve fit and kinetic parameters k_{cat} and Kₘ were calculated by fitting initial rates to the Michaelis-Menten equation.

### Results and Discussion

The light generating enzymatic reaction of firefly luciferase has been used for the continuous monitoring of ATP forming reactions [18]. The luminescent signal generated is proportional to ATP concentration. By coupling D-luciferin/luciferase to an ATP forming reaction in which adenine is converted to ATP, the sensitive quantification of adenine is accomplished (FIG. 1). For the quantification of ATP, ATPlite, a proprietary formulation of D-luciferin/luciferase with a long luminescence half-life was used. The 2x coupling buffer supports the catalytic activities of APRTase, PPDK, and firefly luciferase while providing a quench to neutral pH for RTA catalytic assays at pH 4.0. In order to reduce anion inhibition of firefly luciferase, this buffer contained acetate and sulfate as counterions for Tris and magnesium salts respectively [19]. Ammonium molybdate was included to inhibit the inorganic pyrophosphatase activity of PPDK [20]. Contamination of adenine, AMP, and ATP in the coupling buffer reagents was removed with an essential charcoal purification step because of a large background signal. After charcoal purification, generally less than 50 femtomoles/assay of nucleo-base contamination comprised the background signal of the 2x activated coupling buffer. This contamination was traced to nucleo-bases present within the purified enzymes APRTase and PPDK.

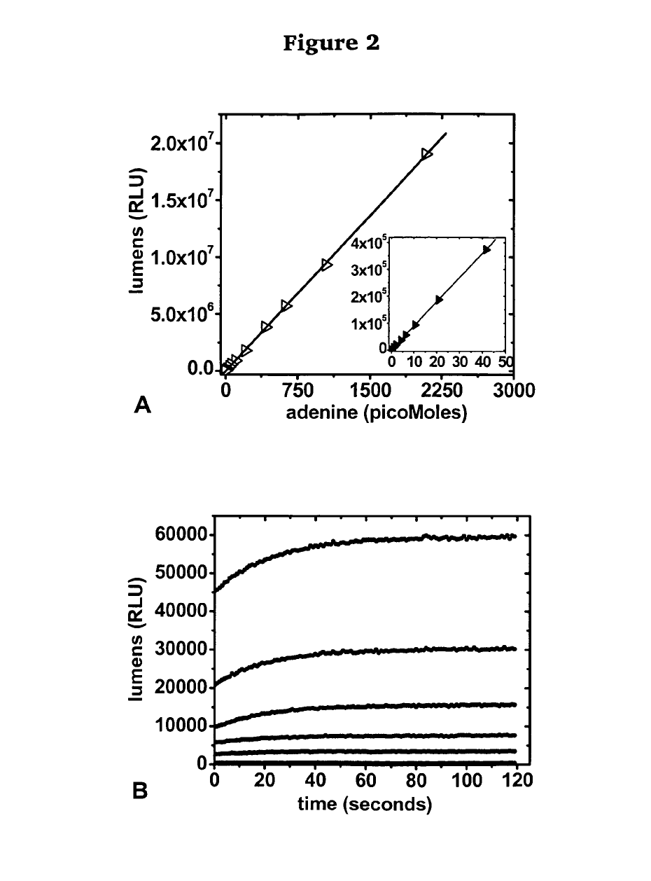

In the discontinuous assay format, adenine standards between 1-2,000 pMoles gave a linear curve fit it relation to luminescence (FIG. 2A). The adenine to ATP conversion reaction occurs to completion as was verified by comparing ATP standard versus luminescence linear fits (data not shown). The continuous assay of adenine was accomplished by combining the D-luciferin/luciferase reagent with the 2x activated coupling buffer. Excess ATPlite was observed to inhibit the adenine to ATP conversion rate and less than optimal amounts reduced the luminescent response for lower concentrations of adenine. A 1:5 (v/v) ratio of ATPlite to 2x activated coupling buffer was observed to be optimal for adenine sensitivity and luminescent rate (data not shown). Assaying increasing concentrations of adenine directly gave a stable proportional increase in luminescence (FIG. 2B). In the continuous adenine-luciferase assays, the concentrations of adenine in standards and released during ribosome catalysis are below saturation k_{cat} for the coupling enzymes and luciferase. Capture of adenine is accomplished by the relatively efficency of APRTase with a k_{cat}/Kₘ of 3.0 M⁻¹ s⁻¹ for APRTase and a similar value of 2.8 M⁻¹ s⁻¹ for PPDK, where the Kₘ values for adenine (APRTase) and AMP (PPDK) are both 9 μM [16, 21]. For firefly luciferase there are two Kₘvalues for ATP, with 111 μM for the initial flash and 20 μM for the continuous lower production of light [22].

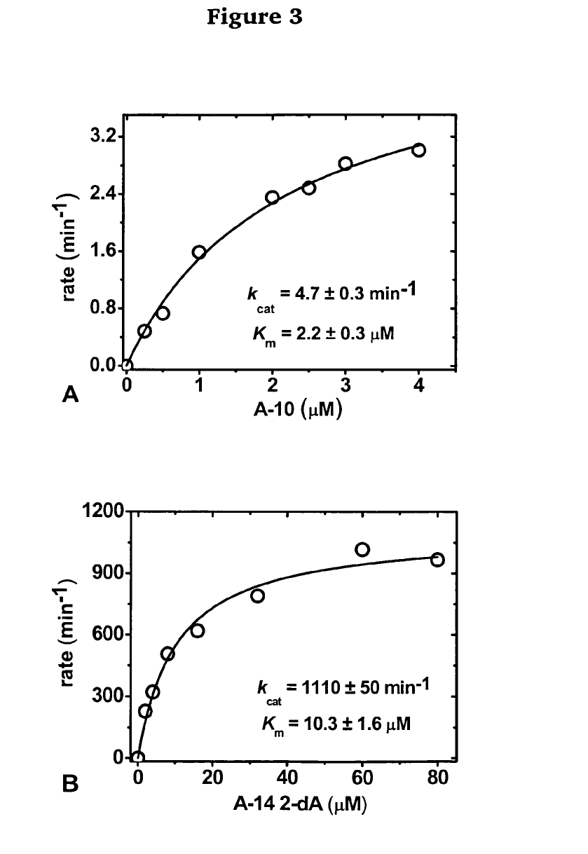

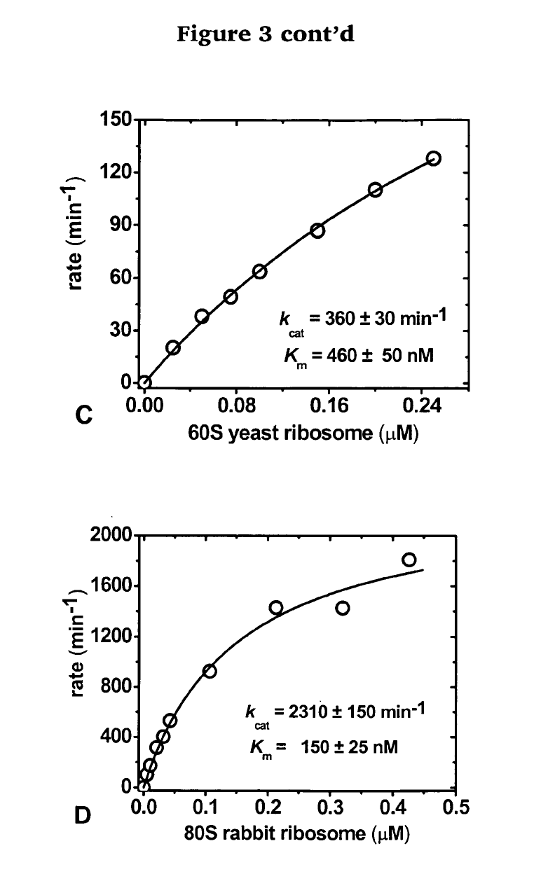

RTA reactions on stem-loop substrates at pH 4.0 were assayed discontinuously and the addition of 2x activated coupling buffer provided a neutral pH quench. RTA initial rate kinetics for the catalysis of stem-loop substrate A-10 measured by the adenine-luciferase assay gave a saturating curve fit with a k_{cat} value of 4.7±0.3 min⁻¹ and a Kₘ of 2.2±0.3 μM (FIG. 3A). Previously determined kinetics for RTA catalysis of A-10 are consistent with these values with a k_{cat} of 4.1 min⁻¹ and a Kₘ of 4.0 μM [8]. With the adenine-luciferase assay, RTA catalysis of A-14 2dA stem-loop RNA/DNA hybrid was measured to give a k_{cat} of 1110±50 min⁻¹ and a Kₘ of 10±1.5μM at pH 4.0. Thus A-14 2dA is a superior substrate for RTA when compared to RNA A-14, which has a reported k_{cat} of 219±14 min⁻¹ and a Kₘ of 8.1±0.7 (FIG. 3B) [8]. Deoxyadenosine at the depurination site (GdAGA) of RNA stem-loop oligonucleotides provides a 5-fold increase in the k_{cat} of catalysis by RTA [13].

RTA catalysis of stem-loop substrates was not detectable at neutral pH and above pH 6.5 no adenine release was observed with either A-10 or A-14 2dA stem-loop oligonucleotides (up to 100 μM) with 2 μM RTA for 10 minutes (data not shown). The kinetic parameters of RTA on truncated stem-loop substrates have been previously characterized with a pH optimum of 4.0 for binding and catalysis using a HPLC separation and detection assay for adenine quantification [8]. Truncated stem-loop substrate mimics of the SRL contain the GAGA tetra-loop motif and GC base pairs to form a stable stem region and to properly fold the tetra-loop for RTA recognition. Other type I and II RIPs have a similar low pH catalytic optimum on nucleic acid substrates such as poly(A) and hsDNA, while the natural target ribosome substrate is depurinated optimally at physiologic pH [7, 23]. The pH 4.0 catalytic optimum of RTA on 6 to 18-mer stem-loop DNA and RNA 28S SRL mimic oligonucleotides results from the protonation of two ionizable residues on substrate and/or the RTA [8, 13, 24].

The measurement of initial rates of adenine released from RTA catalysis of ribosomes at pH 7.4 in the discontinuous assay format required a quench step prior to adenine to ATP conversion with 2x activated coupling buffer. RTA reactions were quenched by acidification with excess HCl and subsequently neutralized to pH 7.0 with KOH. Though RTA is active at acidic pH 4.0, this quench step acidified the reaction to a pH<1 and irreversibly inactivated RTA. The depurination of ribosomes by RTA releases a single adenine from each ribosome to provide a 1:1 stoichiometry. Complete depurination of the SRL can therefore be used to determine active ribosome concentration in solutions and during assays. The presence of trace contamination of ribosome preparations by AMP and ATP were problematic for discontinuous ribosome assays. However, this contamination was reduced by the use of a size exclusion spin column. Initial rate kinetics for depurination of 60S yeast ribosomes by RTA at pH 7.4 were fit to the Michaelis-Menten equation to give a k_{cat} of 360±30 min⁻¹ and Kₘ of 460±50 nM at 37° C. (FIG. 3C). With 33 pM RTA as the depurinating toxin, initial rate kinetics of 80S rabbit reticulocyte ribosomes in the discontinuous assay gave a saturating curve fit with a k_{cat} value of 2310±150 min⁻¹ and a Kₘ value of 150±25 nM at 20° C. (FIG. 3D).

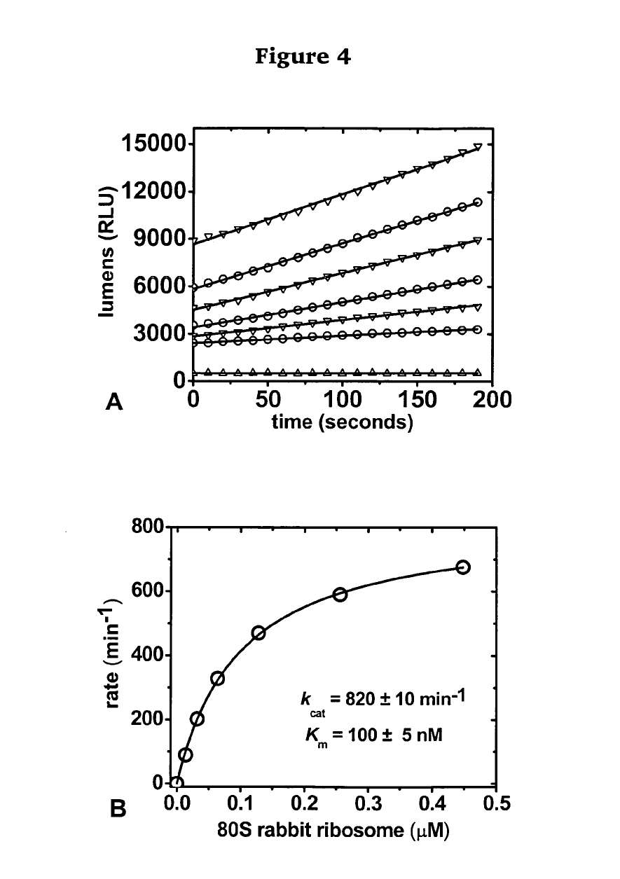

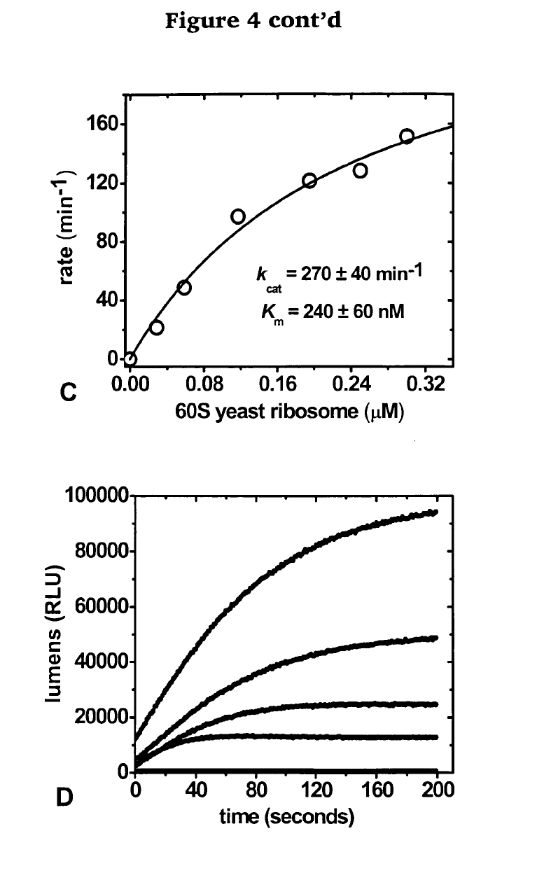

The depurination of 80S rabbit reticulocyte ribosomes by RTA was measured continuously at pH 7.7 by monitoring a linear increase in luminescence with the initial reaction rate being dependent on the ribosome concentration (FIG. 4A). The curve for the initial reaction rate as a function of 80S ribosome concentration fit to the Michaelis-Menten equation with a k_{cat} of 820±10 min⁻¹ and a Kₘ of 100±5 nM (FIG. 4B). Under identical conditions, the k_{cat} and Kₘ values for RTA catalysis of 60S yeast ribosome were 270±40 min⁻¹ and 240±60 nM respectively (FIG. 4C). RTA catalysis of 80S ribosomes in continuous versus discontinuous assay formats gave similar Kₘ values but an ˜3-fold difference in k_{cat} values. This difference is attributed to the difference in buffer conditions for these assays (Table 1). The concentrations of RTA in continuous ribosome reactions can be maintained at very low values to make the production of adenine rate limiting in the overall assay for light production. During these continuous ribosome assays, the concentration of RTA can be maintained as low as 33 pM and 66 pM for 60S and 80S ribosome, respectively, while resulting is readily detected activity by the adenine-luciferase detection assay.

Yeast 60S and rabbit 80S reticulocyte ribosome depurination by RTA was assayed with the adenine-luciferase assay in both discontinuous and continuous formats to yield comparable kinetic parameters (Table 1). RTA has Kₘ values below 500 nM for 60S and 80S ribosome with catalytic rates (k_{cat}) comparable to that for the depurination of deoxyadenosine (GdAGA) stem-loop oligonucleotides A14-2dA which was assayed at pH 4.0 (Table 1). The depurination of 80S rabbit reticulocyte ribosomes by RTA has been reported to give a k_{cat} of ˜1500 min⁻¹ and a Kₘ of 0.1-0.2 μM by aniline RNA degradation analysis [11] and these values are comparable with the adenine-luciferase assay kinetic parameters reported here (Table 1). The action of RTA on 80S rabbit reticulocyte ribosome depurination was ˜3 fold faster than the action on 60S yeast ribosomes while the Kₘ differs by 2-fold under identical temperatures (Table 1).

TABLE 1
RTA kinetic paramaters

| Substrate | Kₘ (μM) | k_{cat} (min⁻¹) |
|---|---|---|
| A-10 ^{d} | 2.2 ± 0.3 | 4.7 ± 0.3 |
| A-14 2dA ^{d} | 10.3 ± 1.6 | 1110 ± 50 |
| 60S yeast ribosome ^{d} | 0.460 ± 0.05 | 360 ± 30 |
| 60S yeast ribosome ^{c}* | 0.240 ± 0.06 | 270 ± 40 |
| 80S rabbit ribosome ^{d}* | 0.150 ± 0.025 | 2310 ± 150 |
| 80S rabbit ribosome ^{c}* | 0.100 ± 0.005 | 820 ± 10 |

^{d} substrate assayed in discontinous format
^{c} substrate assayed in continous format
* substrate assayed at room temparture (20° C.)

The reported k_{cat} for RTA activity on rat liver ribosomes is ˜1800 min⁻¹ with a Kₘ of 2.6 μM at pH 7.6 [10]. In the absence of ribosomal proteins, RTA's hydrolytic rate is reported to be 0.07 min⁻¹ with a similar binding affinity [10]. This dependence on ribosomal proteins suggests that the protein-RNA architecture is an essential element in RTA action on ribosomes. The lack of stem-loop catalysis by RTA under physiologic conditions is reminicient of this change of RTA efficiency on depurinated ribosomes, but the exact chemical mechanism requiring acid pH for small stem-loop depurination remains to be elucidated. Mammalian ribosomal proteins L9 and L10e are near the ribosomal depurination site and may provide structural recognition elements for RTA catalysis [25]. In addition a conformational change in RTA could occur during ribosome recognition as to refold the toxin to bind and depurinate the target SRL [26]. If such an active RTA conformation persisted after contact with the ribosome, it would be expected that RTA could depurinate small stem-loops at neutral pH in the presence of ribosomes. The adenine-luciferase assay was used to determine if small stem-loop catalysis (A-10 and A-14 2-dA) could occur at neutral pH in the presence of ribosomes (60S or 80S). In these experiments, only adenine released from ribosome depurination was observed with RTA (data not shown), establishing that the active configuration for ribosome inactivation by RTA occurs only during ribosomal contact.

By monitoring adenine release from 80S ribosome with the adenine-luciferase assay, femtomole quantities of active RTA can be detected (FIG. 4D). For use as a ricin detection system, contaminating nucleo-bases from samples can be removed with a size exclusion desalting column in a reducing buffer. Potential RNase contamination is slowed by the presence of RNase inhibitor in the 2x activated coupling buffer but can also be readily controlled for by omitting APRTase in the buffer and quantifying ATP formation from RNase-generated AMP [12].

### CONCLUSION

By coupling the light generating enzyme firefly luciferase to an ATP forming reaction in which adenine is converted to ATP, the sensitive quantification of adenine in the RTA depurination reaction for kinetic analysis is demonstrated. Free adenine is converted to ATP in two enzymatic steps using APRTase and PPDK to generate AMP and ATP respectively where the later is quantified by luminescence with D-luciferin/luciferase (FIG. 1). The assay can quantify picomole to nanomole quantities of adenine in a discontinuous assay format and is easily adaptable for high throughput. For continuous adenine assays, APRTase, PPDK, and D-luciferin/luciferase are in a one pot reaction at pH 7.7. A luminescent signal stable for several minutes is observed with a optimized D-luciferin/luciferase formulation along by AMP cycling from PPDK and luciferase enzymes [12]. This detection system provides the advantage of detection only catalytically active ricin and related depurinating toxins since only these forms are relevant in the biological action of the toxin. In contrast, detection systems based on antibiotic and ricin protein binding assays give signals for inactive ricin, leading to the possibility of false positives.

RTA kinetics were investigated on truncated sarcin-ricin loop (SRL) mimics A-10 and A-14 2dA at acidic pH discontinuously where the adenine-luciferase assay provided a neutral pH quench and converted adenine to ATP. Kinetic parameters for 60S yeast and 80S rabbit ribosome were measured using the discontinuous assay at pH 7.4 by including a RTA quench step prior to the adenine-luciferase discontinuous assay. RTA catalysis and initial rate kinetics of 60S and 80S ribosome depurination was also investigated in the continuous adenine-luciferase assay format.

Extreme sensitivity is provided by the luciferase-adenine assay, with as little as 7 femtomole of adenine/assay of RTA being readily detected within minutes by directly measuring adenine release from 80S rabbit ribosome (FIG. 4D). It is envisioned that the buffer conditions and/or D-luciferin/luciferase preparation of the adenine-luciferase assay can be further optimized to enhance the sensitivity of adenine quantification or adapt it to other applications. For assaying larger quantities of adenine (nanomoles to micromoles), the adenine to ATP coupling buffer can be used spectrophotometrically by coupling the pyruvate generated by PPDK to a lactate dehygrogenase/ NADH assay (FIG. 1, materials and methods). The adenine-luciferase assay can also be adapted for high throughput to detect potential RTA inhibitors or the activity based detection of other RIP enzymes to aid in isolation and discovery.

### REFERENCES

1. Franz, D. R. and Jaax, N. K., in Medical aspects of chemical and biological warfare 1997. Chapter 32, 631-642.
2. Nicolson, G. L., Blaustein, J., Etzler M. E., Characterization of Two Plant Lectins from Ricinus communis and Their Quantitative Interaction with a Murine Lymphoma. Biochemistry, 1974. 13, 196-204.
3. Sandvig, K., and van Deurs, B., Endocytosis, intracellular transport, and cytotoxic action of Shiga toxin and ricin. Physiol. Rev., 1996. 76, 949-966.
4. Wesche, J., Rapak, A., and Olsnes, S. Dependence of Ricin Toxicity on Translocation of the Toxin A-chain from the Endoplasmic Reticulum to the Cytosol. J Biol Chem, 1999. 274, 34443-34449.
5. Wälchli, S., Skånland, S. S., Gregers, T. F., Lauvrak, S. U., Torgersen, M. L., Ying, M., Kuroda, S., Maturana, A., and Sandvig, K. EDEM Is Involved in Retrotranslocation of Ricin from the Endoplasmic Reticulum to the Cytosol Mol. Biol. Cell 2006. 17, 1664-1675.
6. Endo Y, Mitsui K, Motizuki M, Tsurugi K. , The mechanism of action of ricin and related toxic lectins on eukaryotic ribosomes. The site and the characteristics of the modification in 28 S ribosomal RNA caused by the toxins J. Biol. Chem., 1987. 262(12): p. 5908-5912.
7. Barbieri L, Valbonesi P, Bonora E, Gorini P, Bolognesi A, Stirpe F. , Polynucleotide: adenosine glycosidase activity of ribosome-inactivating proteins: effect on DNA, RNA and poly(A). Nucleic Acids Research, 1997. 25, 518-522.
8. Chen, X.-Y., Link, T. M., and Schramm, V. L. Ricin A-Chain: Kinetics, Mechanism, and RNA Stem-Loop Inhibitors. Biochemistry, 1998. 37, 11605-11613.

### 9. Heisler I, Keller J, Tauber R, Sutherland M, Fuchs H. A Colorimetric Assay for the Quantitation of Free Adenine Applied to Determine the Enzymatic Activity of Ribosome-Inactivating Proteins Analytical Biochemistry, 2002. 302, 114-122.

10. Endo Y, Tsurugi K., The RNA N-Glycosidase Activity of Ricin A-chain. The characteristics of the enzymatic activity of ricin A-chain with ribosomes and with rRNA The Journal of Biological Chemistry, 1988. 263(18): p. 8735-8739.
11. Olsnes S, Fernandez-Puentes C, Carrasco L, Vazquez D., Ribosome Inactivation by the Toxic Lectins Abrin and Ricin. Eur. J. Biochem., 1975. 60, 281-288.

### 12. Sakakibara T, Murakami S, Eisaki N, Nakajima M, Imai K., An Enzymatic Cycling Method Using Pyruvate Orthophosphate Dikinase and Firefly Luciferase for the Simultaneous Determination of ATP and AMP (RNA). Analytical Biochemistry, 1999. 268, 94-101.

13. Amukele, T. K. and Schramm, V. L., Ricin A-chain substrate specificity in RNA, DNA, and hybrid stem-loop structures. Biochemistry, 2004. 43, 4913-4922.
14. Wang H C, Ciskanik L, Dunaway-Mariano D, von der Saal W, Villafranca J J., Investigations of the Partial Reactions Catalyzed by Pyruvate Phosphate Dikinase. Biochemistry, 1988. 27, 625-633.

### 15. Goss N H, Evans C T, Wood H G. , Pyruvate Phosphate Dikinase: Sequence of the

Histidyl Peptide, the Pyrophosphoryl and Phosphoryl Carrier. Biochemistry, 1980. 19, 5805-5809.

16. Shi W, Tanaka K S, Crother T R, Taylor M W, Almo S C, Schramm V L. Structural Analysis of Adenine Phosphoribosyltransferase from Saccharomyces cerevisiae. Biochemistry, 2001. 40, 10800-10809.

### 17. Wool, I. G., THE STRUCTURE AND FUNCTION OF EUKARYOTIC RIBOSOMES. Annual Reviews of Biochemistry 1979. 48, 719 -754.

18. Lundin A, Richardsson A, Thore A., Continuous Monitoring of ATP-Converting Reactions by Purified Firefly Luciferase. Analytical Biochemistry 1976. 75, 611-620.
19. Nichols W W, Curtis G D, Johnston H H. , Choice of Buffer Anion for the Assay of Adenosine 5′-Triphosphate Using Firefly Luciferase. Anal. Biochem., 1981. 114, 396-397.
20. Ernst S M, Budde R J, Chollet R., Partial Purification and Characterization of Pyruvate, Orthophosphate Dikinase from Rhodospirillum rubrum. J Bacteriol., 1986. 165, 483-488.
21. Ye D, Wei M, McGuire M, Huang K, Kapadia G, Herzberg O, Martin BM, Dunaway-Mariano D., Investigation of the Catalytic Site within the ATP-Grasp Domain of Clostridium symbiosum Pyruvate Phosphate Dikinase. J. Biol. Chem, 2001. 276, 37630-37639.
22. M. DeLuca, and W. D.McElroy, Two kinetically distinguishable ATP sites in firefly luciferase Biochemical and Biophysical Research Communications 1984. 123, 764-770.
23. Barbieri L, Ciani M, Girbés T, Liu W Y, Van Damme E J, Peumans W J, Stirpe F., Enzymatic activity of toxic and non-toxic type 2 ribosome-inactivating proteins. FEBS Letters, 2004. 563, 219-222.
24. Amukele, T. K., Roday, S., and Vern L. Schramm, V. L. Ricin A-chain activity on stem-loop and unstructured DNA substrates. Biochemistry, 2005. 44, 4416 -4425.
25. Vater C A, Bartle L M, Leszyk J D, Lambert J M, Goldmacher V S Ricin A Chain Can Be Chemically Cross-linked to the Mammalian Ribosomal Proteins L9 and L10e. Journal of Biological Chemistry, 1995. 270. 12933-12940.
26. Argent R H, Parrott A M, Day P J, Roberts L M, Stockley P G, Lord J M, Radford S E., Ribosome-mediated Folding of Partially Unfolded Ricin A-chain. J Biol Chem, 2000. 275, 9263-9269.

1. A method for detecting the presence of a compound that catalyzes release of adenine from a nucleic acid, the method comprising:
(a) converting adenine released by catalysis of a nucleic acid by the compound to AMP using adenosine phosphoribosyl transferase, and
(b) converting the AMP formed in step (a) to ATP using pyruvate orthophosphate dikinase,
wherein the presence of ATP is indicative of the presence of the compound.

2. The method of claim 1, wherein the amount of ATP formed in step (b) is proportional to the amount of the compound.

3. The method of claim 1, wherein ATP is detected using a luciferase.

4. The method of claim 3, wherein the luciferase is firefly luciferase.

5. The method of claim 1, wherein the nucleic acid is DNA or RNA.

6. The method of claim 5, wherein the RNA is associated with a ribosome.

7. The method of claim 1, wherein the compound is a ribosome-inactivating protein.

8. The method of claim 7, wherein the ribosome-inactivating protein is ricin.

9. A method for detecting the presence of ricin comprising:
(a) converting adenine released by catalysis of a nucleic acid by ricin to AMP using adenosine phosphoribosyl transferase, and
(b) converting the AMP formed in step (a) to ATP using pyruvate orthophosphate dikinase,
wherein the presence of ATP is indicative of the presence of ricin.

10. The method of claim 9, wherein the amount of ATP formed in step (b) is proportional to the amount of ricin.

11. The method of claim 9, wherein ATP is detected using a luciferase.

12. The method of claim 11, wherein the luciferase is firefly luciferase.

13. The method of claim 9, wherein the nucleic acid is RNA.

14. The method of claim 13, wherein the RNA is associated with a ribosome.

15. The method of claim 9, wherein ricin is detected.

16. A method for screening for an inhibitor of a compound that catalyzes release of adenine from a nucleic acid, the method comprising:
(a) contacting nucleic acid with the compound in the presence and in the absence of a potential inhibitor of the compound;
(b) converting adenine released by catalysis of the nucleic acid by the compound to AMP using adenosine phosphoribosyl transferase;
(c) converting the AMP formed in step (b) to ATP using pyruvate orthophosphate dikinase; and
(d) measuring the amount of ATP formed in step (c),
wherein a reduction in the amount of ATP formed in the presence of the potential inhibitor compared to the amount of ATP formed in the absence of the potential inhibitor indicates that the potential inhibitor is an inhibitor of the compound, or
wherein a lack of reduction in the amount of ATP formed in the presence of the potential inhibitor compared to the amount of ATP formed in the absence of the potential inhibitor indicates that the potential inhibitor is not an inhibitor of the compound.

17. The method of claim 16, wherein the amount of ATP formed in step (c) is proportional to the amount of the compound.

18. The method of claim 16, wherein ATP is detected using a luciferase.

19. The method of claim 18, wherein the luciferase is firefly luciferase.

20. The method of claim 16, wherein the nucleic acid is DNA or RNA.

21. The method of claim 20, wherein the RNA is associated with a ribosome.

22. The method of claim 16, wherein the compound is a ribosome-inactivating protein.

23. The method of claim 22, wherein the ribosome-inactivating protein is ricin.
